(19) United States
(12) Patent Application Publication
(10) Pub. No.: US 2019/0000779 A1
(43) Pub. Date: Jan. 3, 2019

(54) PHARMACEUTICAL FORMULATION

(71) Applicants: Jamie Dukes, Lithonia, GA (US); Kevin Dennis, Franklin, TN (US)

(72) Inventors: Jamie Dukes, Lithonia, GA (US); Kevin Dennis, Franklin, TN (US)

(21) Appl. No.: 15/636,586

(22) Filed: Jun. 28, 2017

(51) Int. Cl. A61K 31/135; A61K 31/375; A61K 31/4415; A61K 31/59; A61K 31/51; A61K 31/519; A61K 9/00

(52) U.S. Cl. CPC A61K 31/135; A61K 31/375; A61K 31/4415; A61K 9/0029; A61K 31/51; A61K 31/519; A61K 9/0019; A61K 31/59

(57) ABSTRACT

A method for the treatment of chronic pain and nutritional replacement is provided. In some implementations, the method consisting of parenteral administration to a patient of ketamine bolus in a dosage amount effective to treat pain followed by a parenteral administration to the patient of a composition comprising an effective amount ketamine, Vitamin C, Vitamin B6, and Vitamin D.

### CROSS REFERENCE TO RELATED APPLICATION

This application claims the benefit of U.S. Patent Application Ser. No. 62/355,813, which was filed on Jun. 28, 2016, and is incorporated herein by reference in its entirety.

### TECHNICAL FIELD

This disclosure relates to implementations of a pharmaceutical formulation.

### BACKGROUND

There are many patients who suffer from intractable pain syndromes that emerge from ineffective pain management. Chronic pain is a long standing problem in healthcare that has risen to epidemic levels. Standard therapies that have been in existence for the last 20 years are being challenged as addictive failures. Opioid and narcotic deaths from overdoses are at 46 patients deaths per day and is considered to be the highest death rate in American history. Intractable pain is one of the most debilitating symptoms cancer patients suffer during radiation and chemotherapy. It is believed by some that cancer patients are at the highest risk for opioid resistance, addiction, and intractable pain syndromes. Multiple medications are often combined to provide a multi system “attack” on pain centers in cancer patients as pain tolerance is reduced.

### DETAILED DESCRIPTION

Implementations of a pharmaceutical formulation used to manage pain in humans are provided. In some implementations, the pharmaceutical formulation may include nutrients (e.g., vitamins B⁶, C, and D,) that reduce the proliferation and/or metastasis of cancer cells in humans.

Two implementations (Infusion A and Infusion B) of the pharmaceutical formulation are listed below. Infusion A and infusion B are parenteral formulations. In this way, the infusions may be intravenously administered.

Infusion A comprises:

Normal Saline: 0.9% solution;

Ketamine: 0.3 mg\kg\IV Bolus over 10 min, with subsequent 0.1-0.3 mg\kg\hr (max dose 15 mg);

Magnesium Sulfate: 2 gm\IV;

Vitamin C (ascorbate): 0.5 gm\kg\IV;

Vitamin B⁶: 2 gm\IV; and

Vitamin D: 4,000 IU\IV.

Infusion B comprises:

Normal Saline 0.9% solution;

Magnesium Sulfate: 2 gm\IV;

Vitamin C (ascorbate) : 0.5 gm\kg\IV;

Vitamin B⁶: 2 gm\IV; and

Vitamin D: 4,000 IU\IV.

In some implementations, the Infusion A and Infusion B pharmaceutical formulations of the present disclosure may further comprise Thiamine (B1): 100 mg/IV and Folate: 5 mg/IV.

In some implementations, the Infusion A and Infusion B pharmaceutical formulations of the present disclosure may be prescribed as adjunct treatments to chemotherapy and radiation in oncology patients to assist in cancer cell reduction and pain control.

In some implementations, the Infusion A and Infusion B pharmaceutical formulations of the present disclosure may be administered by intravenous route over a period of 30 minutes to 60 minutes for chronic pain and nutritional replacement.

In some implementations, the Infusion A pharmaceutical formulation may be administered following an initial intravenous Ketamine bolus at 0.3 mg/kg of Bolus intravenous ketamine over 10 minutes. Thereafter, 0.1-0.3 mg\kg\hr Ketamine along with the other ingredients may be administered. This regimen provides optimal pain relief for the patient during the Infusion A infusion. In some implementations, the maximum Ketamine dosing IV for safety is set at <=15 mg/hr.

In some implementations, an Infusion C pharmaceutical formulation may comprise Ketamine HCL 1%, Magnesium Sulfate 4%, Ascorbic Acid (Vit C) 70%, Pyridoxine (Vit B6) 4%, Vitamin D 0.0002% (4000IU), and Sodium Chloride (0.9%) 50 ml. In some implementations, the Infusion C pharmaceutical formulation may further comprise thiamine 0.4%.

In some implementations, an Infusion D pharmaceutical formulation may comprise Magnesium Sulfate 4%, Ascorbic Acid (Vit C) 70%, Pyridoxine (Vit B6) 4%, Vitamin D 0.0002% (4000IU), and Sodium Chloride (0.9%) 50 ml. In some implementations, the Infusion D pharmaceutical formulation may further comprise thiamine 0.4%.

In some implementations, the pharmaceutical formulations of the present disclosure may be infused twice weekly for maximum nutritional benefit and/or needed pain control. In some implementations, the pharmaceutical formulations of the present disclosure may be infused less than twice weekly for maximum nutritional benefit and/or needed pain control. In some implementations, the pharmaceutical formulations of the present disclosure may be infused more than twice weekly for maximum nutritional benefit and/or needed pain control.

In some implementations, the pharmaceutical formulations of the present disclosure may be used in the treatment of oncological chronic neuropathic pain and nutritional replacement.

In some implementations, a pharmaceutical formulation comprising the ingredients of any of the pharmaceutical formulations of the present disclosure may be prescribed to treat chemo-induced encephalopathy. In some implementations, such a pharmaceutical formulation may further comprise thiamine (B1). In some implementations, memantine may be administered orally prior to administering such a pharmaceutical formulation. In some implementations, memantine may be administered orally 30 minutes prior to administering such a pharmaceutical formulation. In some implementations, memantine may be administered orally less than 30 minutes prior to administering such a pharmaceutical formulation. In some implementations, memantine may be administered orally more than 30 minutes prior to administering such a pharmaceutical formulation.

In some implementations, a pharmaceutical formulation may consist of any of the above-disclosed formulations.

Reference throughout this specification to “an embodiment” or “implementation” or words of similar import means that a particular described feature, structure, or characteristic is included in at least one embodiment of the present invention. Thus, the phrase “in some implementations” or a phrase of similar import in various places throughout this specification does not necessarily refer to the same embodiment.

Many modifications and other embodiments of the inventions set forth herein will come to mind to one skilled in the art to which these inventions pertain having the benefit of the teachings presented in the foregoing descriptions and the associated drawings.

The described features, structures, or characteristics may be combined in any suitable manner in one or more embodiments. In the above description, numerous specific details are provided for a thorough understanding of embodiments of the invention. One skilled in the relevant art will recognize, however, that embodiments of the invention can be practiced without one or more of the specific details, or with other methods, components, materials, etc. In other instances, well-known structures, materials, or operations may not be shown or described in detail.

1. A method for the treatment of chronic pain and nutritional replacement, the method consisting of:
parenteral administration to a patient of ketamine bolus in a dosage amount effective to treat pain; and
then followed by a parenteral administration to the patient of a composition comprising an effective amount ketamine, Vitamin C, Vitamin B⁶, and Vitamin D.

2. The method of claim 1 wherein the ketamine bolus is administered for at least 10 minutes.

3. The method of claim 1 wherein the composition further comprises thiamine and folate.

4. The method of claim 1 wherein said parenteral administrations are performed twice weekly.

5. A method for the treatment of chronic pain and nutritional replacement, the method consisting of:
parenteral administration to a patient of ketamine bolus at 0.3 mg/kg of Bolus intravenous ketamine; and
then followed by a parenteral administration to the patient of a composition comprising 0.1-0.3 mg\kg\hr of ketamin, 0.5 gm\kg\IV of Vitamin C, 2 gm\IV of Vitamin B⁶, and 4,000 IU\IV of Vitamin D.

6. The method of claim 5 wherein the ketamine bolus is administered for at least 10 minutes.

7. The method of claim 5 wherein the composition further comprises 100 mg/IV of thiamine and 5 mg/IV of folate.

8. The method of claim 5 wherein said parenteral administrations are performed twice weekly.

9. A method for the treatment of chemo-induced encephalopathy, the method consisting of:
oral administration to a patient of an effective amount of memantine; and
then parenteral administration to the patient of ketamine bolus in a dosage amount effective to treat pain; and
then followed by a parenteral administration to the patient of a composition comprising an effective amount ketamine, Vitamin C, Vitamin B⁶, and Vitamin D.

10. The method of claim 9 wherein the composition further comprises thiamine.

11. The method of claim 9 wherein the memantine is administered at least 30 minutes prior to parenteral administration to the patient of ketamine bolus.

12. The method of claim 11 wherein the ketamine bolus is administered for at least 10 minutes.
